(19) United States
(12) Patent Application Publication
(10) Pub. No.: US 2005/0232984 A1
(43) Pub. Date: Oct. 20, 2005

(54) Non-vesicular cationic lipid formulations

(76) Inventors: Heinrich Haas, Munchen (DE); Toralf Peymann, Munchen (DE)

Correspondence Address: MORGAN LEWIS & BOCKIUS LLP, 1111 PENNSYLVANIA AVENUE NW, WASHINGTON, DC, 20004, US

(21) Appl. No.: 10/525,384

(22) PCT Filed: Aug. 25, 2003
(86) PCT No.: PCT/EP03/09398

§ 371 Date: Feb. 23, 2005

(30) Foreign Application Priority Data
Aug. 23, 2002 (EP) 02018907.2
Aug. 23, 2002 (EP) EP0306760

(51) Int. Cl. A61K 9/127

(52) U.S. Cl. 424/450

(57) ABSTRACT

The present invention relates to a non-vesicular preparation comprising at least one cationic amphiphile in an aqueous environment, its production and use and a cationic liposome suspension obtainable thereof with increase drug trap ratio and its areas of application such as pharmacology and medicine, particularly its use as carrier system for active substances.

The present invention relates to a non-vesicular preparation comprising at least one cationic amphiphile in an aqueous environment, its production and use and a cationic liposome suspension obtainable thereof with increased drug trap ratio and its areas of application such as pharmacology and medicine, particularly its use as carrier system for active substances.

Liposomes play a significant role in medical and pharmaceutical sciences as drug delivery systems. In a typical application, an active compound, if it is lipopohilic, is encapsulated in the bilayer lipid membrane of the liposome or, if it is hydrophilic, is inserted into the aqueous compartment, in order to have it delivered to a target site.

For the preparation of liposomes a variety of well-known methods are available (R. R. C. New (ed.) Liposomes, A Practical Approach, Oxford University Press, Oxford 1990). However, liposomes which comprise water-soluble compounds, and which fulfil the requirements of homogeneity, narrow size distribution with small liposome sizes, as well as high drug to lipid values are still difficult to achieve. High drug to lipid ratios however, are of particular importance for medical applications.

With the usual standard methods for liposome formation, the encapsulation efficacy for water-soluble compounds is low. For example, a compound can be loaded on basis of the well known film method: A thin film of lipid on the inner wall of a flask is reconstituted with an aqueous solution, which contains the compound to be encapsulated. The fraction of the compound which is enclosed in the so-formed liposomes corresponds to the fraction of encapsulated with respect to the total volume. Common liposome formulations have concentrations in the range from 10-50 mM with liposome diameters in the range from 100 to 300 nm. For such formulations, the ratio of encapsulated to total volume is small and therefore the encapsulation efficacy is small. Most of the compound remains in the free aqueous phase and is usually removed by dialysis. This has the further disadvantage that most of the valuable compound is lost.

The non-encapsulated compound is removed, since it may cause side effects if it is not protected in the liposomal carrier. Further, it may have pharmacokinetic characteristics which are different to those of the liposomal drug. In case of targeted delivery by the liposomes, the non-liposomal fraction of the compound is inactive. For this reasons it is important to minimize the non-liposomal fraction of the drug.

A variety of methods has been described to overcome this intrinsic problem of encapsulation of compounds in the aqueous compartment of liposomes. One of it is the active loading technique, which is applicable to compounds where the membrane permeability can be different, for example as a function of the pH value (R. R. C. New (ed.) Liposomes, A Practical Approach, Oxford University Press, Oxford 1990). In that case, by applying a pH gradient from the inner to the other side of the liposome, the compound can be trapped in the vesicle. However, these approaches are applicable only to a limited number of suitable molecules and to particular environmental conditions. Therefore, so far none of them provided a substantial general breakthrough for liposomal formulations of water soluble compounds.

In WO 96/05808 and WO 99/49716 a method for producing concentrated ‘vesicular phospholipid gels’ by using high-pressure homogenisation is disclosed. These semi-solid phospholipid pastes or -gels with high lipid content consist predominantly of vesicular structures (WO 96/05808, WO 99/49716 and Brandl 2001 (M. Brandl (2001) Liposomes as drug carriers: a technological approach, Biotechnology annual review Volume 759-85). WO 96/05808 discloses liposome preparations from unilamellar vesicles of small and medium size (100-300 nm), with high/drug ratios of at least 20% w/w. However, several disadvantages are linked to that approach: The preparation is highly viscous, and re-dispersion is done best under rigorous mechanical stress, such as an oscillating bath mill which is a disadvantage for delicate materials. As well WO 99/49716 refers to liposome gels, with at least 20% of an active compound, wherein the compound is added to the liposome gel and, by heating or mechanical stress, the compound is equally distributed inside and outside the vesicles. However, due to the high viscosity of these liposome gels, and due to the size of the vesicles, sterile filtration, which is an important step during the formation of pharmaceutical preparations, is not possible.

Recently it was reported, that cationic liposomes have high affinity to angiogenic blood vessels around a solid tumor (Schmitt-Sody M. et al. (2003) Clin Cancer Res 9, 2335-41), which makes them useful for specific targeting of a drug to the tumor site (vascular targeting). However, as has been discussed above, many drugs of interest can partition into the aqueous phase. For liposomal formulations of such compounds a certain fraction is present in the free aqueous phase and thus is inactive with respect to the targeting capacity of cationic liposomes.

In general, for compounds which have a certain solubility in water or which have a high permeability across the membrane, the loading of the liposome with the drug is a problem which has not been sufficiently solved so far. In all presently available approaches a significant fraction of the compound is not encapsulated. It is not active in the sense of specific targeting of the carrier. It may be removed by dialysis or equivalent techniques, but a significant amount will be lost. Another difficulty is, that the encapsulated state is usually a non-equilibrium state, since in the thermodynamic equilibrium the compound is uniformly distributed. Therefore, depending on the membrane permeability of the compound, during the time between dialysis and application further material may be released from the liposome into the aqueous phase.

The problem underlying the present invention was to provide an improved drug delivery and/or release system with a high drug to lipid ratio, target specificity and sufficient stability for pharmaceutical application.

Thus, the solution to the above problem is achieved according to the invention by providing the embodiments characterized in the claims.

The invention relates to a non-vesicular preparation comprising at least one cationic amphiphile in the range of about 10 mM to about 600 mM, preferably of about 25 mM to about 500 mM, more preferably of about 100 mM to about 400 mM, and most preferably of about 200 mM to about 300 mM, optionally a further amphiphile in the range of about up to 60 mol % with respect to the total amphiphile concentration and optionally a stabilizing agent in the range of about 10 mM to about 600 mM, preferably of about 100 mM to about 500 mM and more preferably of about 200 mM to about 400 mM.

Unexpectedly, it was found, that a clear transparent phase which is virtually free of light scattering particles and which is not a dispersion of liposomes or any other particulate dispersion can be obtained if cationic amphiphiles, preferably lipids are mixed in an aqueous phase. This new phase can be obtained with a wide range of amphiphile concentrations, from about <20 mM up to about >600 mM. It appears that there is no lower concentration limit, and the high concentration limit is close to the state of swollen lipid bilayers with no excess of water.

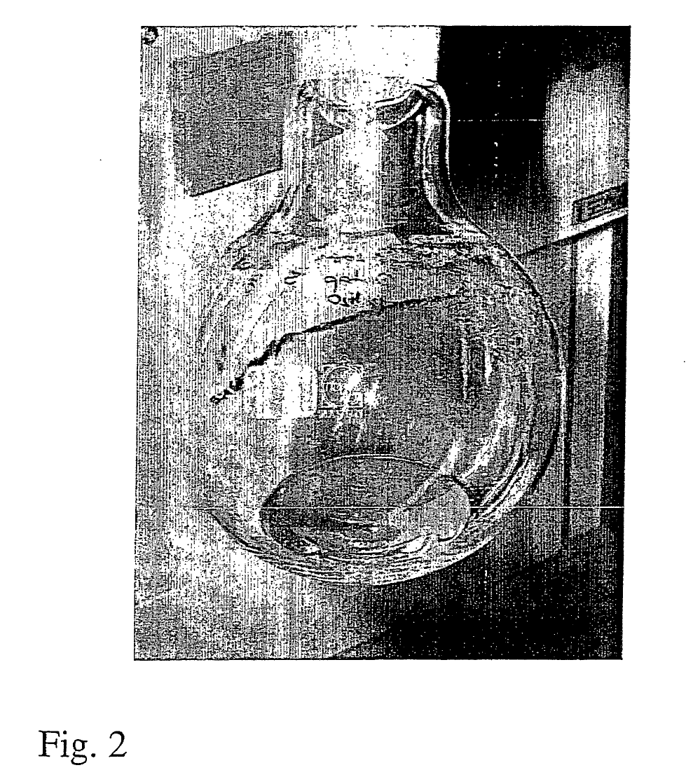

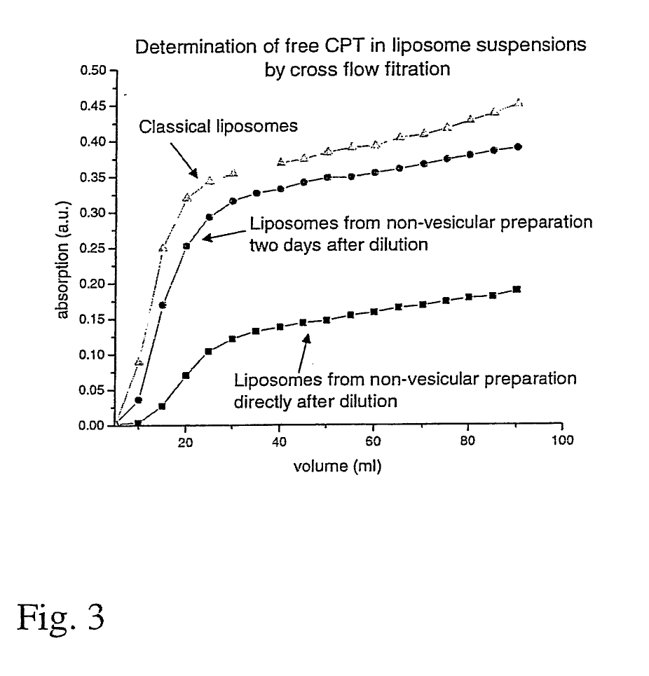

The inventive preparation can be described by being a transparent, isotropic, substantially homogeneous phase which differs in various fundamental aspects from classical liposome suspensions (FIG. 2). As a directly visible attribute, liposome suspensions appear white opalescent due to light scattering from liposome particles. The inventive preparation, to the contrary, is clear and transparent, i.e., virtually no light scattering particles are present. Trials of quasi elastic light scattering measurements (Zetasizer 3000, Malvern, Herrenberg, Germany) indicate that the scattering intensity is reduced by at least a factor of 300 with respect to liposome suspensions with a mean size of about 180 nm. Under usual conditions, liposome suspensions of 1 mM concentrations give a count rate of about 60 kCps. For the inventive preparation of DOTAP at 270 mM a count rate of about 40 kCps is measured. Virtually no size distribution can be determined and virtually no indication for particles >10 nm is found (Malvern Contin analysis).

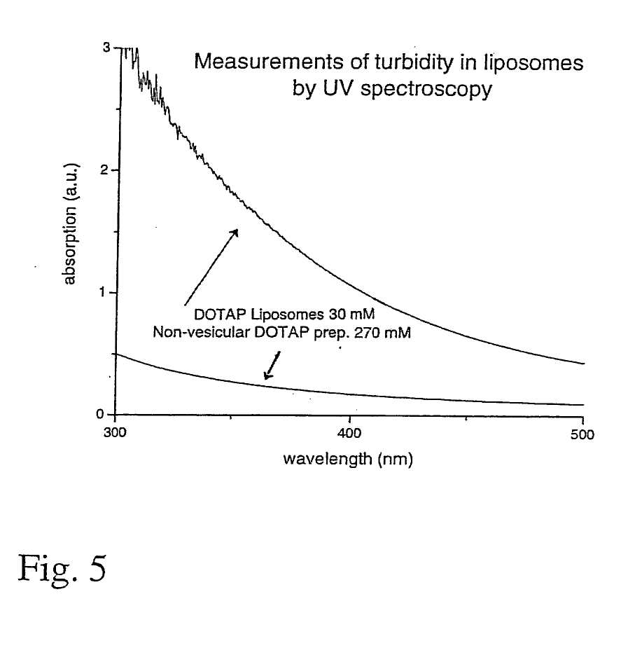

The particle number can also be deduced from turbidity measurements, which can be performed by UV-vis spectroscopy. In FIG. 5 the UV spectra of a 30 mM DOTAP liposome suspension and of a 270 mM non-vesicular preparation of DOTAP are shown. As can be seen, the absorption (and therefore the scattering) is much higher for the liposome suspension as for the non-vesicular preparation, even though the latter has a concentration which is about one order of magnitude higher. Comparison of absorption at a selected wavelength (400 nm) indicates, that the molar scattering of the non vesicular preparation is less than 2% of that of the liposome suspension.

As a further characteristic, the inventive preparation shows low macroscopic viscosity up to rather high lipid concentrations (>200 mM), i.e., visual inspection suggests a liquid like state, similar to that of the aqueous phase since it can be easily extruded through membranes of 200 nm pore size (the pore size which is usually used for sterile filtration). This makes the preparation potentially applicable as a ready to use pharmaceutical composition also for applications in which sterile filtration is demanded, especially if an active compound is present. Compared with this, viscosity of liposome suspensions above a certain concentration (>50 mM) are often too high for extrusion and sterile filtration and thus not suitable for pharmaceutical use.

The inventive preparation is remarkably different from formerly described so-called vesicular liposome gels (WO 96/05808 and WO 99/49716) since gels are solid-like or semi-solid colloidal structures. The named liposome gels are composed of individual lipid vesicles at high packing density. In order to allow a component to migrate into a lipid vesicle, mechanical agitation or elevated temperature is necessary (WO 99/49716). The inventive preparation however, can be described as a homogeneous phase wherein no encapsulated or free aqueous phase can be distinguished. All components in the aqueous phase are free to move across the whole volume. If a further component is added, it can distribute across the whole phase and a uniform mixture can be achieved.

The inventive preparation can be transformed into a liposome suspension by dilution with water or an aqueous solution. Since the inventive preparation can be also produced at low concentrations, this result was unexpected. In fact, by the ‘single phase method’ the inventive preparation can be obtained already at concentrations <25 mM and by subsequent further solvent evaporation it can be concentrated up to more than 600 mM without affecting its physical state. (i.e., it continues to be a clear, transparent phase). It was therefore rather expected that the inventive preparation can be diluted without affecting its molecular state of aggregation. Instead, by the dilution, the molecular organization changes and liposomes are formed.

So-formed liposomes are preferably in the small to medium size range (30-300 nm) with a narrow size distribution (PI values from size measurements by quasi-elastic light scattering >0.5), which makes them applicable for pharmaceutical application. Further, entrapment of a water-soluble active compound in the aqueous compartment of the liposomes (formed by dilution of the preparation as disclosed) is a function of the encapsulated/total volume at the time of liposome formation. If liposome formation occurs at a concentration which is higher than that of the final liposome concentration (which is usually in the range from 10 to 25 mM), e.g. at a concentration of about 100 mM, the resulting trap rate of the obtainable liposomes is higher as can be achieved if the liposomes are formed directly at a low concentration (for example by reconstitution of a lipid film with an aqueous phase which contains the component, see FIG. 3).

Summarizing, the inventive preparation has the following advantages: It is
suitable for direct pharmaceutical use
suitable for loading an active compound
suitable for the preparation of liposomes with a high trap rate and a narrow size distribution.

The present invention might be characterized more specifically by its method of production. Lipid dispersions in water may exist in a large number of different phase and aggregation states, which may be thermodynamically stable or metastable (D. F. Evans, H. Wennerström: The Colloidal Domain: Where Physics Chemistry, Biology and Technology Meet, VHC publishers, Weinheim, 1994). Therefore, by selecting a different mode of preparation a different type of molecular organization in the resulting phase can be obtained. If that phase state is not the thermodynamically most favourable one, nevertheless it can be stable for long time periods, particularly long enough to provide sufficient shelf life for production and storage before an application. On the other hand, a metastable phase may be transformed into a more stable one by applying a suitable stress to the system.

As an example, a procedure will be given to obtain the inventive preparation at a molecular composition, for which by using another procedure, classical liposomes are obtained: a 25 mM dispersion of DOTAP in water can be produced as a classical liposome dispersion, for example if it is produced by the well-known film method or by ethanol injection. If the dispersion is produced by the subsequently described ‘single phase evaporation technique’ however, with the identical molecular composition, the inventive preparation is obtained. The thermodynamically less favourable state is hindered from transforming into the more favourable one by the high energy barrier of such a transition. In order to form or break a liposome, which is the more favourable thermodynamic state, the lipid bilayer must be disrupted, which requires a significant amount of energy.

In general, the inventive preparation can be obtained by several ways, e.g. by mixing water and an organic solvent, in which the amphiphiles are solubilized. By removing the organic solvent, the inventive preparation is formed. Any other technique however, well known in the art which permits to obtain a particle free dispersion of lipid in water by chemical, physical or mechanical means is thereby suitable to produce the inventive preparation. On the other hand all procedures in which the rupture of bilayers and subsequent re-fusion to closed vesicle is involved, i.e., the procedures which are usually applied for liposome production, like the well know film method or ethanol injection, are less favourable, since these can lead to the formation of vesicles can which remain, stable or metastable, in the preparation. Therefore reconstitution of a lipid film to multilamellar vesicles, such as described in WO 96/05808, should be avoided to obtain the inventive preparation.

The inventive preparation comprises cationic amphiphiles, which are selected from lipids, lysolipids or pegylated lipids having a positive net charge. The lipid may comprise several, e.g. two hydrocarbon chains, which are not necessarily identical, which are branched or unbranched, saturated or unsaturated with a mean chain length from C12 to C24. Preferred are cationic lipids with at least one tertiary amino or quaternary ammonium group.

Useful lipids for the present invention include:
DDAB, dimethyldioctadecyl ammonium bromide; N-[1-(2,3-dioleoyloxy)propyl]-N,N,N-trimethyl ammonium methylsulfate (DOTAP); 1,2-diacyloxy-3-trimethylammonium propanes, (including but not limited to: dioleoyl, dimyristoyl, dilauroyl, dipalmitoyl and distearoyl; also two different acyl chain can be linked to the glycerol backbone); N-[1-(2,3-dioloyloxy)propyl]-N,N-dimethyl amine (DODAP); 1,2-diacyloxy-3-dimethylammonium propanes, (including but not limited to: dioleoyl, dimyristoyl, dilauroyl, dipaimitoyl and distearoyl; also two different acyl chain can be linked to the glycerol backbone); N-[1-(2,3-dioleyloxy)propyl]-N,N,N-trimethylammonium chloride (DOTMA); 1,2-dialkyloxy-3-dimethylammonium propanes, (including but not limited to: dioleyl, dimyristyl, dilauryl, dipalmityl and distearyl; also two different alkyl chain can be linked to the glycerol backbone); dioctadecylamidoglycylspermine (DOGS); 3-[N—(N′,N′-dimethylaminoethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA); -alanyl cholesterol; cetyl trimethyl ammonium bromide (CTAB); diC14-amidine; N-tert-butyl-N′-tetradecyl-3-tetradecylaminopropionamidine; 14Dea2; N-(alpha-trimethylammonioacetyl)didodecyl-D-glutamate chloride (TMAG); O,O′-ditetradecanoyl-N-(trimethylammonioacetyl)diethanolamine chloride; 1,3-dioleoyloxy-2-(6-carboxy-spermyl)-propylamide (DOSPER); N,N,N′,N′-tetramethyl-N,N′-bis(2-hydroxylethyl)-2,3-dioleoyloxy-1,4-butanediammonium iodide; 1-[2-(acyloxy)ethyl]2-alkyl(alkenyl)-3-(2-hydroxyethyl)-imidazolinium chloride derivatives as described by Solodin et al. (1995) Biochem. 43:13537-13544, such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM), 2,3-dialkyloxypropyl quaternary ammonium compound derivatives, containing a hydroxyalkyl moiety on the quaternary amine, as described e.g. by Felgner et al. [Felgner et al. J. Biol. Chem. 1994, 269, 2550-2561] such as: 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE); cationic esters of acyl carnitines as reported by Santaniello et al. [U.S. Pat. No. 5,498,633]; cationic triesters of phosphatidylcholine, i.e., 1,2-diacyl-sn-glycerol-3-ethylphosphocholines, where the hydrocarbon chains can be saturated or unsaturated and branched or non-branched with a chain length from C₁₂ to C₂₄, the two acyl chains being not necessarily identical.

In a preferred embodiment the cationic amphiphile is selected from a quaternary ammonium salt such as N-[1-(2,3-diacyloxy)propyl]-N,N,N-trimethyl ammonium, wherein a pharmaceutically acceptable counter anion of the quaternary amino compound is selected from the group consisting of chloride, bromide, fluoride, iodide, nitrate, sulfate, methyl sulfate, phosphate, acetate, benzoate, citrate, glutamate or lactate. Preferably, the cationic lipids are in the liquid crystalline state at room temperature. Examples are lipids where the hydrocarbon chains contain one or more double bonds, where the hydrocarbon chains are branched, or where any other packing mismatch is given, for example due to different chains. Further, in many cases lipids with chains shorter than C14 fulfil the requirement.

The inventive preparation may comprise at least one further amphiphile in an amount of about 0 to about 60 mol %, preferably of about 20 mol % to about 50 mol % and most preferably of about 30 mol % to about 40 mol % based on the total amphiphile concentration.

The further amphiphiles may have a negative and/or neutral net charge (anionic and/or neutral amphiphile). These can be selected from sterols or lipids such as cholesterol, phospholipids, lysolipids, lysophospholipids, sphingolipids or pegylated lipids with a negative or neutral net change. Useful anionic and neutral lipids thereby include: Phosphatidic acid, phosphatidylserine, phosphatidylglycerol, phosphatidylinositol (not limited to a specific sugar), fatty acids, sterols containing a carboxylic acid group, cholesterol, 1,2-diacyl-sn-glycero-3-phosphoethanolamine, including but not limited to dioleoyl (DOPE), 1,2-diacyl-glycero-3-phosphocholines, sphingomyelin. The fatty acids linked to the glycerol backbone are not limited to a specific length or number of double bonds. Phospholipids may also have two different fatty acids. Preferably the further lipids are in the liquid crystalline state at room temperature and they are miscible (i.e. a uniform phase can be formed and no phase separation or domain formation occurs) with the used cationic amphiphile, in the ratio as they are applied.

In a preferred embodiment the neutral amphiphile is phosphatidylcholine.

The preparation may further comprise a stabilizing agent, which is preferably selected from a sugar or a polyvalent alcohol or a combination thereof such as trehalose, maltose, sucrose, glucose, lactose, dextran, mannitol or sorbitol. In a preferred embodiment the stabilizing agent is trehalose or glucose.

The preparation may further comprise an organic solvent, particularly a water-soluble organic solvent, e.g. ethanol in an amount up to about 5% (v/v). Instead of ethanol other alcohols or organic solvents can be used as well. For producing a pharmaceutical composition, organic solvents which are not ethanol may need to be removed. Suitable organic solvents are alcohols, e.g. methanol, ethanol, propanol, isopropanol, or ethylene glycol, ethers, e.g. tetrahydrofuran or diethylether, or halogenated hydrocarbons, e.g. chloroform, or mixtures of these solvents.

Unless defined otherwise, all technical and scientific terms used in this specification shall have the same meaning as commonly understood by persons of ordinary skill in the art to which the present invention pertains. “About” in the context of amount values refers to an average deviation of maximum +/−20%, preferably +/−10% based on the indicated value. For example, an amount of about 30 mol % cationic lipid refers to 30 mol %+/−6 mol % and preferably 30 mol %+/−3 mol % cationic lipid with respect to the total lipid/amphiphile molarity.

“Amphiphile” refers to a molecule, which consists of a water-soluble (hydrophilic) and an oil-soluble (lipophilic) part. Lipids and phospholipids are the most common representatives of amphiphiles. In the text, lipid and amphiphile are used synonymously.

“Angiogenesis associated condition” e.g. refers to different types of cancer, chronic inflammatory diseases, rheumatoid arthritis, dermatitis, psoriasis, wound healing and others.

“Camptothecin” refers to 20(S)-Camptothecine(1H-Pyrano[3′,4′:6,7]indolizino[1,2-b]quinoline-3,14(4H, 12H)-dione, 4-ethyl-4-hydroxy-, (S)—), CAS 7689-03-4. ‘Camptothecin’ or ‘camptothecin drug’ in the present context includes as well the carboxylate form of the drug.

“Camptothecin drug” refers to camptothecin itself or a derivative thereof. A camptothecin derivative is obtained by any chemical derivatization of camptothecin (see structure). A non-limiting list of possible camptothecin drugs is given under: http://dtp.nci.nih.gov as from Aug. 19, 2002. In the sketch of the molecule, the most frequent derivatization sites are outlined as R₁-R₅.

Structure of camptothecin drugs:

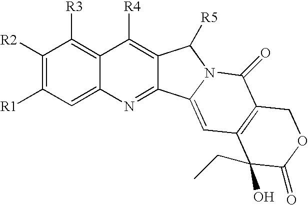

In the following table, typical examples for derivatization at different sites are listed. Camptothecin may be present as a hydrochloride. The lactone ring (E-ring) may be seven-membered instead of six-membered (homocamptothecins).

| Name | R1 | R2 | R3 | R4 | R5 |
|---|---|---|---|---|---|
| Camptothecin | H | H | H | H | H |
| 9-Nitro- | H | H | NO₂ | H | H |
| camptothecin | | | | | |
| 9-Amino- | H | H | NH₂ | H | H |
| camptothecin | | | | | |
| 10-Hydroxy- | H | OH | H | H | H |
| camptothecin | | | | | |
| Topotecan | H | OH | —CH₂—N—(CH₃)₂ | H | H |
| SN38 | H | OH | H | CH₂—CH₃ | H |
| Camptosar ®(Irinotecan) | H | 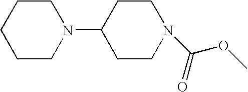 | H | CH₂—CH₃ | H |
| Lurtotecan ® | R1 and R2 is: | | H | H | H |
| O—CH2—CH2—O | | | | | |
| DX-8951f | F | CH₃ | R₃ and R₄ is: | | H |
| | | | —CH2— | | |

Derivatization can influence the properties of CPT to make the molecule more hydrophilic or more lipophilic, or that the lactone-carboxylate equilibrium is affected. In the context of the application of CPT as an anti-cancer drug, derivatization is intended to maintain or to increase activity.

“Cancer” refers to the more common forms of cancers such as bladder cancer, breast cancer, colorectal cancer, endometrial cancer, head and neck cancer, leukaemia, lung cancer, lymphoma, melanoma, non-small-cell lung cancer, ovarian cancer, prostate cancer and to childhood cancers such as brain stem glioma, cerebellar astrocytoma, cerebral astrocytoma, ependymoma, Ewing's sarcoma/family of tumors, germ cell tumor, extracranial, hodgkin's disease, leukemia, acute lymphoblastic, leukemia, acute myeloid, liver cancer, medulloblastoma, neuroblastoma, non-hodgkin's lymphoma, osteosarcoma/malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, Wilms' Tumor and other childhood kidney tumors and to less common cancers including acute lymphocytic leukaemia, adult acute myeloid leukaemia, adult non-hodgkin's lymphoma, brain tumor, cervical cancer, childhood cancers, childhood sarcoma, chronic lymphocytic leukaemia, chronic myeloid leukaemia, esophageal cancer, hairy cell leukaemia, kidney cancer, liver cancer, multiple myeloma, neuroblastoma, oral cancer, pancreatic cancer, primary central nervous system lymphoma, skin cancer, small-cell lung cancer.

“Carrier” refers to a diluent, adjuvant, excipient, or vehicle which is suitable for administering a diagnostic or therapeutic agent. The term also refers to a pharmaceutically acceptable component(s) that contains, complexes or is otherwise associated with an agent to facilitate the transport of such an agent to its intended target site. Carriers include those known in the art, such as liposomes, polymers, lipid complexes, serum albumin, antibodies, cyclodextrins and dextrans, chelates, or other supramolecular assemblies.

“Cationic” refers to an agent that has a net positive charge or positive zeta potential under the respective environmental conditions. In the present invention, it is referred to environments where the pH ist in the range between 3 and 9, preferably between 5 and 8.

“Cationic amphiphiles” as used herein refer to cationic lipids as defined.

“Cationic liposome” refers to a liposome which has a positive net charge. In the present invention, it is referred to environments where the pH is in the range between 3 and 9, preferably between 5 and 8. The cationic liposomes are prepared from the cationic lipids or amphiphiles themselves or in admixture with other amphiphiles, particularly neutral or anionic lipids.

“Derivative” refers to a compound derived from some other compound while maintaining its general structural features. Derivatives may be obtained for example by chemical functionalization or derivatization.

“Drug” as used herein refers to a pharmaceutically acceptable pharmacologically active substance, physiologically active substances and/or substances for diagnosis use.

“Encapsulation efficiency” refers to the fraction of a compound which is encapsulated into the liposomes of a liposome suspension by a given method.

“Homogenization” refers to a physical process that achieves a uniform distribution between several components. One example is high-pressure homogenisation.

“Lipid” in its conventional sense refers to a generic term encompassing fats, lipids, alcohol-ether-soluble constituents of protoplasm, which are insoluble in water. Lipids are amphiphilic molecules such as fatty acids, steroids, sterols, phospholipids, glycolipids, sulpholipids, aminolipids, or chromolipids. The term encompasses both naturally occurring and synthetic lipids. In a more general sense, lipids are characterized as amphiphiles, i.e., they are molecules which consist of lipophilic as well as hydrophilic moieties. Preferred lipids in connection with the present invention comprise at least two alkyl chains with at least 12 carbon chains and are: steroids and sterol, particularly cholesterol, phospholipids, including phosphatidyl and phosphatidylcholines and phosphatidylethanolamines, and sphingomyelins. Fatty acids could be about 12-24 carbon chains in length, containing up to 6 double bonds, and linked to the backbone. The hydrocarbon chains can be different (asymmetric), or there may be only 1 fatty acid chain present, e.g., lysolecithins. Mixed formulations are also possible, particularly if non-cationic lipids are derived from natural sources, such as lecithins (phosphatidylcholines) purified from egg yolk, bovine heart, brain, or liver, or soybean.

“Uposome” refers to a microscopic spherical membrane-enclosed vesicle (about 50-2000 nm diameter) made artificially in the laboratory. The term “liposome” encompasses any compartment enclosed by a lipid bilayer. Liposomes are also referred to as lipid vesicles.

“Lysolipid” refers to a lipid where one fatty acid ester has been cleaved resulting in a glycerol backbone bearing one free hydroxyl group.

“Lysophospholipid” refers to a phospholipid where one fatty acid ester has been cleaved resulting in a glycerol backbone bearing one free hydroxyl group.

“Negatively charged lipids” refer to lipids that have a negative net charge. In the present invention, it is referred to environments where the pH is in the range between 3 and 9, preferably between 5 and 8. Examples are phosphatidic acid, phosphatidylserine, phosphatidylglycerol, phosphatidylinositol (not limited to a specific sugar), fatty acids, sterols.

“Neutral lipids” refer to lipids that have a neutral net charge such as cholesterol, 1,2-diacyl-sn-glycero-3-phosphoethanolamine, including but not limited to dioleoyl (DOPE), 1,2-diacyl-glycero-3-phosphocholines, Sphingomyelin. In the present invention, it is referred to environments where the pH is in the range between 3 and 9, preferably between 5 and 8.

“Non-vesicular cationic preparation” as used herein refers to a composition comprising at least one cationic amphiphile in an aqueous environment. The overall net charge of the amphiphiles is positive, also if further anionic or neutral amphiphiles are present.

“Particle diameter” refers to the size of a particle. To experimentally determine particle diameters, dynamic light scattering (DLS) measurements, using Malvern Zetasizer 1000 or 3000 (Malvern, Herrenberg, Germany) were performed. For quantitative data analysis (determination of Z(average) and PI were determined, or, additionally, data analysis with the ‘Contin’ formalism was performed.

“Pegylated lipid” refers to a lipid bearing one ore more polyethylene glycol residues.

“Pharmaceutical composition” refers to a combination of two or more different materials with superior pharmaceutical properties than are possessed by either component.

“Phospholipid” refers to a lipid consisting of a glycerol backbone, a phosphate group and one or more fatty acids wich are bound to the glycerol backbone by ester bonds.

“Positively charged Lipids” refer to a synonym for cationic lipids (for definition see definition of “cationic lipids”). In the present invention, it is referred to environments where the pH is in the range between 3 and 9, preferably between 5 and 8.

“Stabilizing agent” as used herein refers to a compound which is water soluble and favourable for the stability of the inventive preparation.

“Sterol” refers to a steroid alcohol. Steroids are derived from the compound called cyclopentanoperhydrophenanthrene. Well-known examples of sterols include cholesterol, lanosterol, and phytosterol.

“Therapeutic agent” refers to a species that reduces the extent of the pathology of a disease such as cancer. Such a compound may, for example, reduce primary tumor growth and, preferably, the metastatic potential of a cancer. Alternatively, such a compound may reduce tumor vascularity, for example either by decreasing microvessel size or number or by decreasing the blood vessel density ratio.

“Virtually free” of a species refers to as not detectable by HPTLG. “Virtually free of liposomes” refers to a state, where the signal from a given method such as light scattering, which is proportional to the liposome concentration, is less than 5% of the value as it is obtained in a system which has the same molecular composition but consisting of liposomes.

The inventive preparation is a substantially homogeneous phase comprising at least one cationic amphiphile, optionally at least one further amphiphile, optionally a stabilizing agent and optionally an active compound. The active compound can thereby be hydrophilic, lipophilic or amphipathic compound or a mixture of compound and is preferably selected from a therapeutic or a diagnostic agent.

Preferably, a therapeutic agent is present in the range of about 0.1 mol % to about 20 mol % with respect to the total amphiphile concentration preferably in the range of about 1 mol % to about 15 mol % and more preferably in the range of about 3 mol % to about 10 mol %.

The therapeutically active agent may be selected from an anti-inflammatory drug, an anti-cancer drug, an enzymatic drug, an antibiotic substance, an antioxidant, a hormone drug, an angiogenesis inhibiting agent, a smooth muscle cell-proliferation/migration inhibitor, a platelet aggregation inhibitor, a release inhibitor for a chemical mediator, and a proliferation/migration inhibitor for vascular endothelium. Specific examples are selected from taxanes, from other agents interacting with microtubuli such as epothilones, discodermolide, laulimalide, isolaulimalide, eleutherobin, colchicines and derivatives thereof, vinca alkaloids such as vinorelbine, from platinum complexes such as oxaliplatin, from camptothecins, from anthracyclines such as doxorubicin or from statins (e.g., lovastatin).

More preferably the inventive preparation comprises camptothecin, a camptothecin drug or a derivative thereof in the range of about 0.1 mol % to about 20 mol %, preferably in the range of about 1 mol % to about 15 mol % and more preferably in the range of about 3 mol % to about 10 mol % with respect to total amphiphile concentration.

In a further embodiment, the active compound is selected from diagnostic agents such as imaging agents, e.g. magnetic resonance imaging agents (gadolinium complexes such as Magnevist, Omniscan and others), X-ray and computed tomography contrast agents (compounds with heavy elements with a large number of electrons such as iodine, barium, dysprosium and others; examples include ionic and non-ionic derivatives of iodinated benzoic acid derivatives such as iopamidol and iodixanol, barium sulfate and others), and other agents employed in other imaging modalities (ultrasound, fluorescence, near infrared and others).

Preferably a diagnostic agent such as an imaging agent is present in the range of about 0.1 mol % to about 50 mol %, preferably in the range of about 10 mol % to about 50 mol % and more preferably in the range of about 30 mol % to about 50 mol % with respect to total amphiphile concentration.

As has been disclosed above, unexpectedly, after dilution with an aqueous solution, a suspension of liposomes may be obtained from the inventive preparation. Thus, in a further aspect the present invention relates to a cationic liposome suspension obtainable from the non-vesicular preparation as disclosed.

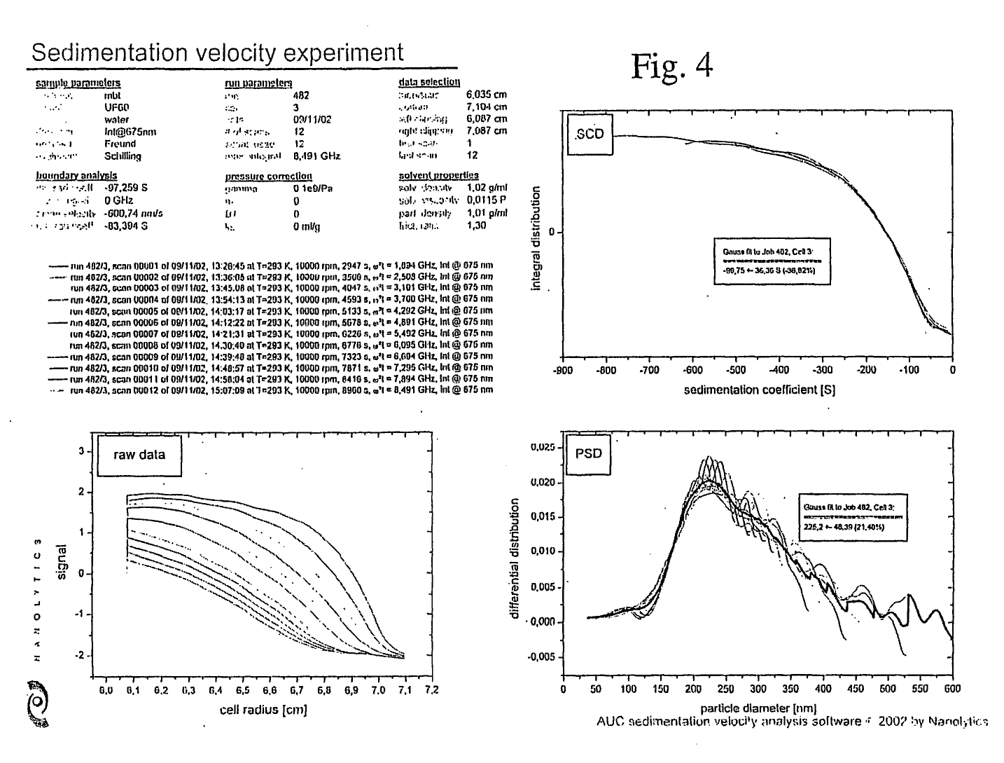

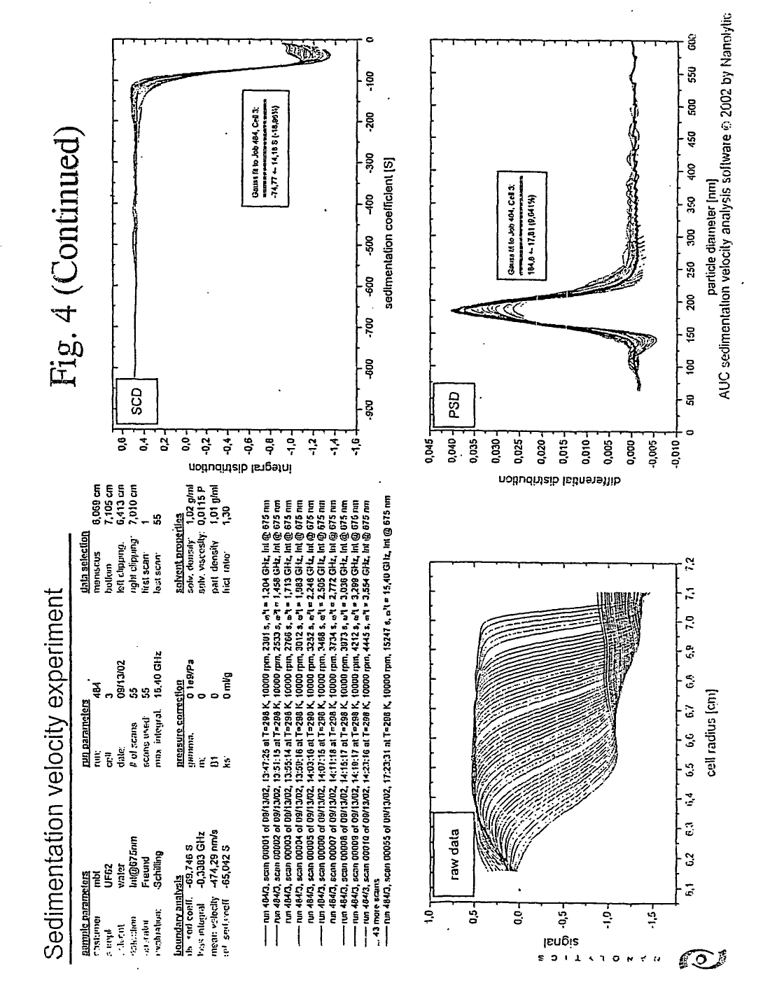

Unexpectedly, so-formed liposomes are characterized by a well-defined size distribution. For example, after dilution of a preparation comprising about 280 mM DOTAP and 2.5 mM CPT to a final DOTAP concentration of 25 mM, size measurement by quasi-elastic light scattering indicate a Z_{average} of 70 nm and a PI value of 0.4. In FIG. 4, results from analytical ultracentrifugation measurements are given. A very narrow size distribution was obtained.

In a preferred embodiment the inventive cationic liposome suspension comprises liposomes of a defined size distribution in the range between about 50 nm to about 1000 nm and in a more preferred embodiment liposomes with a size distribution of about 50 nm to about 500 nm, preferably of about 50 nm to about 300 nm. The small liposome size with well defined size distribution makes the suspension particularly suitable for direct pharmaceutical administration.

Further, the liposome suspension may comprise the liposomally loaded compound in a higher amount as can be obtained with methods state of the art, i.e., the liposomes are ‘overloaded’ with the compound. The liposomes are produced from the inventive preparation by dilution. The maximum gain which is theoretically obtainable can be estimated on basis of a simple calculation: If the preparation is formed at 100 mM total amphiphile concentration, and the final liposome concentration is 10 mM, the fraction of free active compound is reduced by a factor of about ten compared with liposome formulations produced by standard techniques such as lipid film or ethanol injection method.

The present invention is suitable for pharmaceutical application. Accordingly, the present invention provides a pharmaceutical composition comprising the inventive preparation or the cationic liposome suspension as disclosed, optionally together with a pharmaceutically acceptable carrier, diluent and/or adjuvant.

If a purely water-soluble active agent is present at the time of dilution, it is enclosed into the aqueous compartment of the liposome to a higher fraction as if liposomes are formed by classical techniques. If a water-soluble compound can partition into the membrane bilayer, its trap rate in the membrane after dilution will be higher than its equilibrium state at the same concentration. Unexpectedly the release of such a compound from the membrane into the free aqueous phase can occur slowly enough to enable pharmacological administration and thus, the above described vascular targeting effect can be achieved with higher efficiency as with liposomal formulations disclosed in the prior art.

For many liposomal formulations the hydrophilic compound is released from the liposome with a certain time constant. This is particularly the case, if membrane permeability of the compound is high. In many cases the release is too fast to enable production and storage with sufficient shelf life before administration. It is an advantage of the present invention, that the liposome suspension or a pharmaceutical composition obtainable thereof can be provided directly before use. If the inventive preparation, already comprising an active compound, is stored in the concentrated state only a very low fraction is released into the free aqueous phase, since the relative volume of the aqueous phase is small. If it may not have sufficient shelf life, the inventive preparation and active compound can be stored separately, and mixed and diluted directly before use. In this way, formulations with a lifetime, which is usually very low can be provided for a pharmaceutical application. For example, if an encapsulated water-soluble compound is released from the liposome within a time scale of days or even several hours, it cannot be stored before application. Even if such a formulation is prepared directly before use by a classical method, the non-encapsulated fraction must be removed in a time consuming procedure. With the inventive approach, the compound and the concentrated non-vesicular preparation can be mixed, optionally sterile filtrated, and reconstituted to a liposome suspension with a high encapsulation ratio directly before pharmaceutical application, and the liposome suspension can be used directly after dilution. Therefore, even loaded liposome suspensions with a short half life of hours can be provided for application on a regular basis.

Thus, another aspect of the present invention relates to a kit comprising the inventive preparation and an aqueous solution of an active compound as disclosed.

Camptothecin carboxylate is a compound, which is water soluble, but it partitions in cationic lipid membranes due to favourable interactions with cationic lipids. In order to maximize the liposomal fraction, it is desirable to maximize the lipid concentration. However, for practical applications, too high liposome concentrations are disadvantageous, for example due to the high viscosity.

By using a concentrated non-vesicular preparation comprising cationic amphiphiles, preferably lipids and camptothecin, liposomes can be formed, wherein the liposomal fraction corresponds to the concentrated state directly after dilution, i.e., it is temporarily higher than the equilibrium state after dilution. The equilibrium is reached only after few hours, and therefore, if liposomes are prepared from the inventive non-vesicular preparation and applied directly after dilution, they will have a higher fraction of liposomal camptothecin and thus a higher efficacy than liposomes in the equilibrium state.

For illustration, in FIG. 4 a liposome suspensions as obtained from the inventive preparation, and a classical liposome suspension, as produced by ethanol injection and extrusion are compared. Both liposome suspensions comprise 22.5 mM DOTAP and 2.5 mM camptothecin. Using the inventive preparation, a non-vesicular phase comprising 450 mM DOTAP and 50 mM camptothecin was diluted to a concentration of 22.5 mM DOTAP. Then 10 ml of both suspensions were diluted 1:10, and from the resulting 100 ml the free camptothecin was removed by cross-flow filtration. In the course of filtration, the aqueous phase comprising all molecularly dissolved compounds can pass across the membrane. The filtrate was aliquoted in volumina of 5 ml and the amount of free CPT was determined by UV-vis spectroscopy. In FIG. 4, the absorption in the filtrate is shown for the liposomes as obtained from the inventive preparation directly after dilution, the same after two days and, for comparison, the results of a normal liposome suspension. As can be seen, directly after dilution the fraction of free CPT is by about a factor of two lower than after two days. The values for the free camptothecin after two days are still slightly lower that with the classically produced liposomes which might indicate that the equilibrium was still not reached. Generally, with the DOTAP/camptothecin system, the equilibrium is reached after several hours.

The inventive non-vesicular preparation can be produced by a variety of methods, such as outlined in the experimental descriptions.

In a further embodiment, the present invention relates to a method of producing the non-vesicular preparation comprising cationic amphiphiles as disclosed. As has been outlined, the mode of preparation is fundamental to achieve the inventive preparation. For one and the same molecular composition, several metastable phase and aggregation states can occur. Even though these states are thermodynamically metastable, they may be stable in a certain time scale and thus stable enough for production and storage with sufficient shelf life for pharmaceutical applications. By application of external stress, which can be by addition of a component, change of pH, mechanical stress, heating or any other environmental condition, one phase may be transformed into another, thermodynamically more favourable one. On the other hand, in order to keep the system in a certain metatstable phase, it is preferred to avoid such stress.

For production of the inventive preparation preferably but not exclusively at low lipid concentrations (<100 mM) it is favourable to run through a state of a homogeneous lipid solution, for example as a mixture of ethanol and water. Such a preparation can be obtained e.g. by simple mixing an ethanolic lipid solution (about 1 mM to less than about 100 mM) with water or an aqueous solution, optionally comprising further components. Ethanol and optionally partly water is subsequently removed by evaporation and a clear dispersion of lipid in the aqueous phase is obtained (“single phase evaporation method”). The evaporation can occur up to any value with respect to the initial volume, provided there is excess water left in the preparation.

More specifically, the cationic lipid concentration, preferably DOTAP in enthanol ca be from about 0.5 mM to about 50 mM, more preferably from about 1 mM to about 25 mM. The ethanol to water ratio can be in the range from about 1:20 up to about 20:1, preferably from about 1:10 up to about 10:1 and more preferably from about 1:5 up to about 5:1. The final concentration can be any concentration below swollen lipid bilayers with no excess of water, more preferably from about 100 mM to about 600 mM, more preferably from about 200 to about 400 mM.

Instead of lipids amphiphiles as defined may be used and instead of ethanol any suitable organic solvent which is miscible in water such as methanol, ethanol, propanol, isopropanol, ethylene glycol, tetrahydrofuran, chloroform or diethylether or a mixture of these.

With this procedure, no liposomes are formed. Even though a liposome suspension may be thermodynamically more favourable, the eventual formation of liposomes is avoided since the energy barrier of formation of closed bilayer vesicle is too high if no sufficient mechanical, thermal or other stress is applied.

To the contrary, in standard liposome preparation procedures a step in which mechanical, chemical or other stress is applied to the system in order to provide sufficient energy to rupture the bilayer membrane to form the closed vesicles. For example, in the film method this is done by shaking the thin film of swollen lipid bilayers with water, and in ethanol injection by the fast dilution of the highly concentrated ethanol solution in water.

Another possibility for the formation of the inventive preparation, particularly at high concentration (>100 mM) is high pressure homogenisation. Dry amphiphiles, preferably lipids and the aqueous phase are added to the homogenizer without further treatment. Particularly, it is not necessary and not desirable to run through a step of a multilamellar liposome suspension, such as in WO99/49716 and WO96/05808 disclosed. Thus, it is necessary to initially avoid any kind of stress in order to avoid the formation of liposomes.

It is a further object of the present invention referring to a method of producing the non-vesicular preparation comprising at least one cationic amphiphile, comprising the steps of
(a) providing said cationic amphiphile, optionally a further amphiphile, optionally a stabilizing agent, optionally an active compound and an aqueous phase and
(b) subjecting the components of step a) to conditions so that an isotropic, transparent and substantially homogeneous preparation is formed.

Step b) therein may comprise the ‘single phase evaporation’ or high pressure homogenisation method.

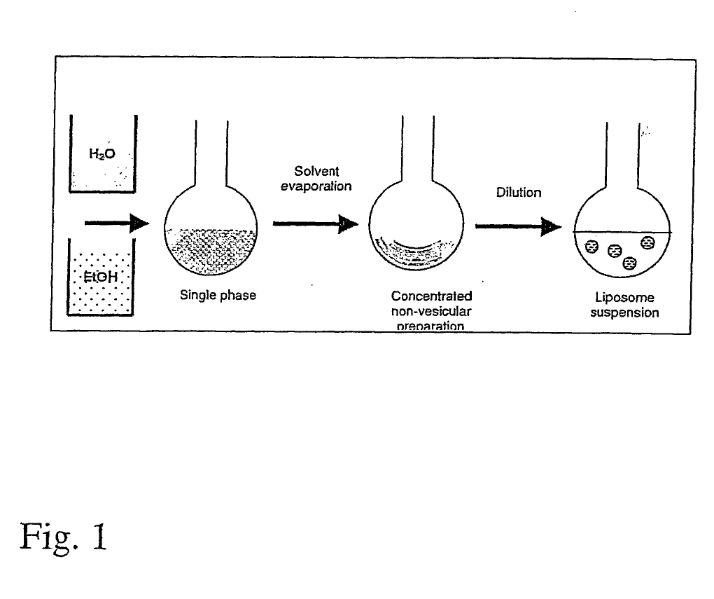

Preferably, the non-vesicular preparation is prepared by mixing a solution of amphiphiles in an organic solvent with an aqueous phase and subsequently removing the organic solvent and optionally water to the desired final concentration (FIG. 1). In this way, the inventive preparation can be obtained at concentrations of up to the limit of swollen lipid bilayers, i.e., when no additional water except of that binding to the lipid headgroups is present.

However, any other technique suitable for the formation of a uniform particle free state can be used for producing the inventive preparation, for example such as given in (D. F. Evans, H. Wennerström: The Colloidal Domain: Where Physics Chemistry, Biology and Technology Meet, VHC publishers, Weinheim, 1994)

As has been disclosed above, the inventive preparation may further comprise an active compound. Advantageously, the active compound can be simply either mixed with the amphiphiles for producing the present preparation if it is lipophilic, or it can be in the aqueous phase, if it is water soluble. Alternatively, the active compound can be added to an already formed preparation. If an active compound, dissolved in water, is added to the already formed inventive preparation, it may freely distribute across the whole phase. A lipophilic compound may be added in dry form and further high pressure homogenisation cycles are applied for homogeneous distribution in the lipid phase.

Since the inventive preparation is not organized in defined closed vesicles, the homogeneous distribution of the added compound is greatly facilitated. Each added compound can distribute homogeneously in the whole phase, and after dilution the active compound is finally encapsulated or inserted into the liposomal membrane. The fraction of the active compound, which is loaded into the liposome is thereby higher as if the formulation was prepared directly at low lipid concentration by a standard liposome forming technique as has been outlined above. Thus, liposomal formulations comprising an active compound can be prepared, wherein the liposomal encapsulated fraction of the water-soluble active compound is increased with respect to the equilibrium state.

The inventive preparation comprising cationic lipids and an active compound can be taken without further dilution as a ready to use pharmaceutical preparation. Its low viscosity up to high concentration enables sterile filtration or extrusion through membranes of defined pore size such as with 100 nm or 200 nm pores, which is a prerequisite for in vivo applications.

As has been disclosed above, the present invention is suitable for the preparation of a medicament or a diagnostic formulation. Thus, it is a further object of the present invention that a preparation, a suspension or a pharmaceutical composition as disclosed can be used for the preparation of a medicament or a diagnostic formulation, particularly for the preparation of a medicament or a diagnostic formulation useful for an angiogenesis associated condition such as an angiogenesis associated disease.

An angiogenesis associated disease is dependent on blood supply. The local interruption of the vasculature will produce an avalanche of cell death. The vascular endothelium is in direct contact with the blood. It is contemplated that a variety of diseases can be prevented and treated with the foregoing methods and compositions. In a preferred embodiment, a preparation, a liposome suspension or a pharmaceutical composition as provided by the present invention may be useful for preventing and/or treating a disease such as cancer, a variety of inflammatory diseases, diabetic retinopathy, rheumatoid arthritis, inflammation, dermatitis psoriasis, stomach ulcers, macular degeneration, hematogenous and solid tumors. In a further preferred embodiment, preparations and compositions of the present invention can be applied for producing a medicament for preventing and/or treating solid tumors and their metastases such as bladder, brain, breast, cervical, colorectal, endometrial, head and neck or kidney cancer, leukemia, liver or lung cancer, lymphoma, melanoma, non-small-cell lung, ovarian, pancreatic or prostate cancer.

The preparation of the present invention may be applied directly or after dilution by injection (e.g. s.c., i.m., i.p.) or implantation. It is also possible to place it into body cavities or to apply it topically onto mucosa, the cornea, or parts of the skin. Thus preparation thus serves as a carrier of the active compound and is responsible for the modified or controlled release of the active compound. Upon transfer into a freely flowing liposome suspension. This suspension may be applied directly by injection (e.g. s.c., i.m., i.p.) or implantation. It is also possible to place it into body cavities or to apply it topically onto mucosa, the cornea, or parts of the skin. The entrapping liposomes lead to a distribution of the active substance carried by the liposomes in the body, which distribution selectively effects a high and long lasting concentration of the active compound at the target site, such is an activated endothelial cell, and thus to an improvement of the effect or to an improvement of the ratio of effect and side effect, or of the therapeutic or diagnostic index.

Figure Legends:

FIG. 1 Scheme for producing the inventive preparation by the single phase solvent evaporation: A diluted solution of (cationic) amphiphiles, preferably lipids and an aqueous solution comprising other components (optionally an active compound) are mixed to form a uniform phase. The organic solvent, preferably ethanol and, optionally, part of the water are evaporated until the desired concentration is reached. The preparation remains as a clear transparent non-vesicular phase. After dilution of the concentrated preparation, liposomes are formed.

FIG. 2 Concentrated preparation containing DOTAP in water at a concentration of about 250 mg/g (w/w). The preparation is water-clear and liquid like.

FIG. 3 Measurements of free camptothecin (CPT) in different liposome formulations. A non-vesicular preparation of 450 mM DOTAP and 50 mM CPT was diluted to a 23.5 mM DOTAP and 2.5 mM CPT liposome suspension. Directly after liposome formation 10 ml of the suspension were further diluted 1:10 and cross-flow filtration was performed. Aliquots of 5 ml of the filtrate were taken and UV-vis measurements were preformed to determine free CPT. In the graph the absorption at 369 nm is shown. Form the same 23.5 mM DOTAP liposome suspension, further 10 ml were diluted after two days, when the system was expected to be at equilibrium. As can be seen, in that case the release is about twice the value as directly after dilution. For comparison, a 23.5 mM DOTAP and 2.5 mM CPT liposome suspension was produced directly by ethanol injection. 10 ml of the extruded (200 nm) liposome formulation were diluted 1:10 and investigated the same way. As can be seen, the values for the free CPT are in the same range as for the suspension from dilution of the non-vesicular preparation after two days.

FIG. 4 Analytical ultracentrifugation measurements for determining the size distribution in liposome formulations. Measurements were performed with 2.5 mM DOTAP and 0.25 mM CPT each. In the upper graph the results from the measurement of classical liposome formulation as prepared by ethanol injection and extrusion (UF60) to a total concentration of 25 mM are shown. For the measurement the sample was diluted 1:10. The lower graph gives the results from a measurement with liposomes as obtained from a non-vesicular preparation at a total concentration of 500 mM (UF62) after dilution of 1:200. The size distribution of the sample from dilution of the non vesicular preparation is rather narrow and even better defined that the one of the extruded liposomes.

FIG, 5 UV-Vis spectroscopy measurements comparing the turbidity of liposome suspensions and the inventive non-vesicular preparation. 30 mM DOTAP liposomes (extruded at 200 nm) and a non-vesicular preparation of 270 mM DOTAP were measured. The absorption from the liposome suspension is much higher that that of the non-vesicular preparation, event though the latter is almost by a factor of ten more concentrated. Quantitative analysis (400 nm) indicates that the molar absorption (due to scattering) of the liposome suspension is more than 50 times higher than that of the non-vesicular preparation.

The following examples should be illustrative only but are not meant to be limiting to the scope of the invention. Other generic and specific configurations will be apparent to those skilled in the art.

### EXAMPLES

### Example 1

A: Non-Vesicular Preparation of DOTAP in Water at High Concentration (Single Phase Evaporation)

33 ml of an ethanolic DOTAP solution, c=6 mM and 10 ml of a 0.5% aqueous solution of trehalose were mixed in a round flask. A clear solution was obtained. The solvent was evaporated at 40° C. at a pressure of 100 mbar until the weight of the solution in the flask was 690 mg. The concentrate was a clear homogeneous phase, without indication for the presence of scattering particles. Density of the preparation was about 1 g/ml, the resulting DOTAP concentration was about 290 mM and the resulting trehalose concentration was about 7%.

B: Formation of a Liposome Suspension by Dilution

The concentrated preparation of part A was diluted with about 7 ml of 10% aqueous trehalose solution to a final concentration of about 25 mM DOTAP. After dilution the clear phase transformed into an opalescent liposome suspension. The size of the liposomes was measured by quasi elastic light scattering measurements (Zetasizer 300, Malvern, Herrenbereg, Germany), Zₐᵥₑ 152 nm.

### Example 2

Non-Vesicular Preparation of DOTAP at Various Concentrations in the Range from 25 mM to 400 mM (Single Phase Evaporation)

All preparations were formed using a solution of DOTAP (DOTAP-CI) in ethanol, c=25 mM and a solution of 10% trehalose in water. For the production of the DOTAP preparations with c=25 mM, 100 mM, 200 mM, 300 mM und 400 mM the equivalent volumina which are necessary to obtain the desired final concentrations and aq. trehalose solutions and water were mixed, such as given in the table.

From the solutions solvent was evaporated until a final volume of about 0.5 ml was obtained. All concentrates were present as water-clear phases.

| c (mM) | V_{Dotap.} (ml) | V_{Trehalose} (ml) | V_{H2O} (μl) |
|---|---|---|---|
| 25 | 0.5 | 0.5 | 22 |
| 100 | 2.0 | 0.5 | 88 |
| 200 | 4.0 | 0.5 | 176 |
| 300 | 6.0 | 0.5 | 264 |
| 400 | 8.0 | 0.5 | 352 |

### Example 3

Non-Vesicular Preparation of DOTAP in Water at High Concentration (High Pressure Homogenization)

To 8.13 g of DOTAP methyl sulfate 35 ml of water was added. The mixture was transferred into the pressure chamber of a high pressure homogenizer. At 750 bar and room temperature, the suspension was homogenized ten times to result in ˜40 ml of a transparent gel-like 300 mM formulation.

### Example 4

Non-Vesicular Preparation of DOTAP and a Gd Complex in Water at High Concentration (High Pressure Homogenization)

The High Pressure Homogenizer (Gaulin Micron LAB 40) holds 40 ml of sample volume. A sample of 36 ml of 0.5 M Gd complex (Omniscan) and 4.65 g of DOTAP methyl sulfate are suspended in the pressure chamber. The homogenisation procedure (room temperature, 750 bar) is repeated ten-fold to yield the respective material. The experiment is performed with two DOTAP concentrations, 150 and 300 mM.

| DOTAP | Appearance of | | Gd | Volume |
|---|---|---|---|---|
| [mM] | Homogenate | Stability | [mM] | increase |
| 150 | homogenous fluid | no precipitation at room | 9 | 3.4 |
| | | temperature | | |
| 300 | homogenous fluid | no precipitation at room | 17 | 2.3 |
| | | temperature, viscid after | | |
| | | dialysis | | |

After homogenisation a homogenous fluid preparation is obtained and extruded through a polycarbonate membrane with 200 nm pore size. The obtained preparation is dialyzed four times against 5% glucose to remove the non-entrapped contrast agent Omniscan. During dialysis the volume of the solution in the dialysis tube increases between 2.3 and 3.4 fold. This increase is taken into account to establish the labelling efficiency. The 300 mM solution turns into a viscous non-vesicular phase during this dialysis. The encapsulation efficiency after dialysis is 6.1% for 150 mM DOTAP and 7.8% for 300 mM DOTAP.

### Example 5

A: Concentrated Non-Vesicular DOTAP/CPT Preparation: DOTAP 500 mM, CPT 50 mM (Single Phase Evaporation)

Ethanolic solution of DOTAP (6 mM) was added to an aqueous solution of CPT-carboxylate (c=2 mM) in 0.5% trehalose with 1% Tris/HCl-buffer, pH 7.5. The solvent was evaporated (30° C. and 25 mbar) to a total concentration of 500 mM DOTAP and 50 mM camptothecin.

B: Formation of a DOTAP/CPT Liposome Suspension by Dilution and Determination of the Overloading Directly After Dilution

The clear concentrated preparation of part A was diluted to a DOTAP concentration of 1 mM (1:500). After dilution an opalescent liposomes suspension was formed.

The fraction of free, non-liposomal, CPT was determined by ‘cross flow filtration’ across a membrane of 50 kDa MWCO. Free CPT was determined directly after dilution and after two days. After dilution the fraction of free CPT was 10% and two days later it was 20%. It is assumed, that the state after two days is the equilibrium state. This indicates, that the fraction of free CPT was reduced by a factor of two directly after dilution.

### Example 6

Adding of CPT to a Pre-Formed Non-Vesicular Concentrated DOTAP Preparation

To 5 ml of a 280 mM non-vesicular preparation of DOTAP in water as obtained from high pressure homogenization (see Example 3) 5 ml of a solution of a 14 mM solution of CPT carboxylate in water was added. A clear, slightly yellowish phase was obtained.

1 ml of the preparation were diluted with a 10 mM Tris/HCl buffer, pH 7.5 to a final concentration of 15 mM.

### Example 7

Tolerability of Liposomes from a Non Vesicular DOTAP/CP Preparation in Mice.

A non-vesicular preparation, DOTAP 450 mM camptothecin 25 mM, was reconstituted with an aqueous solution of 10% trehalose to a liposome suspension of about 25 mM (dilution 1:20). Directly after dilution, the mice were treated with a singe injection of 5 μmol/g. The injections were well tolerated, no adverse effects were observed.

### Example 8

Human Therapy Treatment Protocols

This example is concerned with human treatment protocols using the preparations and suspensions disclosed. Treatment will be of use for diagnosing and/or treating various human conditions and disorders associated with enhanced angiogenic activity. It is considered to be particularly useful in anti-tumor therapy, for example, in treating patients with solid tumors and hematological malignancies or in therapy against a variety of chronic inflammatory diseases such as psoriasis.

A feature of the invention is that several classes of diseases and/or abnormalities are treated without directly treating the tissue involved in the abnormality e.g., by inhibiting angiogenesis the blood supply to a tumor is cut off and the tumor is killed without directly treating the tumor cells in any manner.

Methods of treating such patients using lipid:drug complexes have already been formulated. It is contemplated that such methods may be straightforwardly adapted for use with the method described herein. As discussed above, other therapeutic agents could be administered either simultaneously or at distinct times. One may therefore employ either a pre-mixed pharmacological composition or “cocktail” of the therapeutic agents, or alternatively, employ distinct aliquots of the agents from separate containers.

The various elements of conducting a clinical trial, including patient treatment and monitoring, will be known to those of skill in the art in light of the present disclosure.

For regulatory approval purposes, it is contemplated that patients chosen for a study would have failed to respond to at least one course of conventional therapy and would have objectively measurable disease as determined by physical examination, laboratory techniques, or radiographic procedures. Such patients would also have no history of cardiac or renal disease and any chemotherapy should be stopped at least 2 weeks before entry into the study.

The required application volume is calculated from the patient's body weight and the dose schedule. Prior to application, the formulation can be reconstituted in an aqueous solution. Again, the required application volume is calculated from the patient's body weight and the dose schedule.

The disclosed formulations may be administered over a short infusion time. The infusion given at any dose level should be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses should be administered to groups of patients until approximately 60% of patients showed unacceptable Grade III or IV toxicity in any category. Doses that are ⅔ of this value would be defined as the safe dose.

Physical examination, tumor measurements, and laboratory tests should, of course, be performed before treatment and at intervals of about 3-4 weeks later. Laboratory tests should include complete blood counts, serum creatinine, creatine kinase, electrolytes, urea, nitrogen, SGOT, bilirubin, albumin, and total serum protein.

Clinical responses may be defined by acceptable measure or changes in laboratory values e.g. tumormarkers. For example, a complete response may be defined by the disappearance of all measurable disease for at least a month. Whereas a partial response may be defined by a 50% or greater reduction.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Administration and Dosing

The present invention includes a method of delivery of a pharmaceutically effective amount of the inventive preparation or liposome suspension obtainable thereof comprising an active compound to an angiogenic vascular target site of a subject in need thereof. A “subject in need thereof” thereby refers to a mammal, e.g. a human.

The route of administration comprises peritoneal, parenteral or topic administration and the formulations are easily administered in a variety of dosage forms such as implantation depots, injectable solutions and the like. For use with the present invention the term “pharmacologically effective amount” of a compound administered to a subject in need thereof (which may be any animal with a circulatory system with endothelial cells which undergo angiogenesis) will vary depending on a wide range of factors. For example, it would be necessary to provide substantially larger doses to humans than to smaller animal. The amount of the compound will depend upon the size, age, sex, weight, and condition of the patient as well as the potency of the substance being administered. Having indicated that there is considerable variability in terms of dosing, it is believed that those skilled in the art can, using the present disclosure, readily determine appropriate dosing by first administering extremely small amounts and incrementally increasing the dose until the desired results are obtained. Although the amount of the dose will vary greatly based on factors as described above, in general, the present invention makes it possible to administer substantially smaller amounts of any substance as compared with delivery systems which target the surrounding tissue e.g., target the tumor cells themselves.

The pharmaceutically effective amount of a therapeutic agent as disclosed herein depends on the kind and the type of action of the agent. For the examples mentioned here, it is within the range of about 0.1 to about 20 mg/kg in humans.

The pharmaceutically effective amount of a diagnostic agent as disclosed herein depends on the type of diagnostic agent. The exact dose depends on the molecular weight of the compound, and on the type and the intensity of the signal to be detected. For the examples as given here (fluorescein as fluorescence dye, gadolinium complexes as MRI markers), the applied dose may range from about 0.1 to 20 mg/kg. Most frequent doses are in the order of about 5 mg/kg.

1-22. (canceled)

23. A non-vesicular preparation comprising at least one cationic amphiphile in a concentration of about 10 mM to about 600 mM with a mean chain length from C12 to C24, optionally at least one further amphiphile of up to about 60 mol % based on the total amphiphile concentration and optionally at least one stabilizing agent in a concentration of about 10 mM to about 600 mM in an aqueous phase, wherein said preparation is characterized by being transparent, isotropic and substantially homogeneous.

24. The preparation of claim 23, comprising at least one cationic amphiphile in a concentration of about 25 mM to about 500 mM, preferably in a concentration of about about 100 mM to about 400 mM and most preferably in a concentration of about 200 mM to about 300 mM.

25. The preparation of claim 23, comprising a stabilizing agent in a concentration of about 100 mM to about 500 mM, preferably in a concentration of about about 200 mM to about 400 mM.

26. The preparation of claim 23, wherein said cationic amphiphile is selected from lipids, lysolipids, pegylated lipids having a positive net charge.

27. The preparation of claim 26, wherein said cationic amphiphile is selected from cationic lipids with at least one tertiary amino or quaternary ammonium group such as N-[1-(2,3-diacyloxy)propyl]-N,N-dimethylamine or N-[1-(2,3-diacyloxy)propyl]-N,N,N-trimethyl ammonium.

28. The preparation of claim 23, wherein said further amphiphile has a negative or a neutral net charge.

29. The preparation of claim 23, wherein said further amphiphile is selected from sterols or lipids such as cholesterol, phospholipids, lysolipids, lysophospholipids, sphingolipids or pegylated lipids with a negative or neutral net change.

30. The preparation of claim 27, wherein the neutral amphiphile is diacylphosphatidylcholine.

31. The preparation of claim 23, wherein said stabilizing agent is selected from a sugar or an alcohol or a combination thereof such as trehalose, maltose, sucrose, glucose, lactose, dextran, mannitol or sorbitol.

32. The preparation of claim 31, wherein said stabilizing agent is trehalose or glucose.

33. The preparation of claim 23, further comprising an active compound, wherein said active compound may be hydrophilic, hydrophobic or amphipathic.

34. The preparation of claim 33, wherein said compound is a therapeutic agent, preferably camptothecin or a derivative thereof, a taxane or an other microtubuli interacting agent such as an epothilone, discodermolide, laulimalide, isolaulimalide, eleutherobin, colchicine and/or a derivative thereof, a vinca alkaloid such as vinorelbine, a platinum complex such as oxaliplatin, an anthracycline such as doxorubicin or a statin (e.g., lovastatin) and more preferably camptothecin or a derivative thereof in its carboxylate form.

35. The preparation of claim 34, wherein said therapeutic agent is in the range of about 0.1 mol % to about 20 mol %, preferably in the range of about 1 mol % to about 15 mol % and more preferably in the range of about 3 mol % to about 10 mol % based on the total amphiphile concentration.

36. The preparation of claim 33, wherein said compound is a diagnostic agent, preferably an imaging agent.

37. The preparation of claim 36, wherein said diagnostic agent is in the range of about 0.1 mol % to about 50 mol %, preferably in the range of about 10 mol % to about 50 mol % and more preferably in the range of about 30 mol % to about 50 mol % based on the total amphiphile concentration.

38. A method of producing a liposome suspension comprising using a preparation of claim 23 to form a liposome suspension.

39. A method of producing a liposome suspension from the preparation of claim 23 by diluting said preparation with an aqueous solution.

40. A Pharmaceutical composition comprising the preparation of claim 23, optionally together with a pharmaceutically acceptable carrier, diluent and/or adjuvant

41. A method of preparing a medicament or a diagnostic formulation comprising using a preparation of claim 23 to produce a medicament or diagnostic formulation.

42. A method of treating angiogenesis associated condition such as cancer, chronic or acute inflammatory diseases, rheumatoid arthritis, dermatitis, psoriasis or wound healing comprising administering a pharmaceutical composition of claim 40.

43. A method of producing the non-vesicular preparation of claim 23, comprising:
(a) providing
i) said cationic amphiphile, optionally said further amphiphile, optionally said stabilizing agent, optionally said active compound, and
ii) an aqueous phase; and
(b) dispersing the components of i) in said aqueous phase of ii).

44. The method of claim 43, comprising:
(a) providing
i) said cationic amphiphile, optionally said further amphiphile, optionally said stabilizing agent, and
ii) an aqueous solution;
(b) dispersing the components of i) in said aqueous phase of ii); and
(c) adding an active agent to the dispersion of step (b).

45. The method of claim 43, wherein step (b) comprises a single phase evaporation or high pressure homogenisation method.

46. A method of producing the non-vesicular preparation of claim 23, comprising:
a) providing said cationic amphiphile, optionally said further amphiphile, optionally said stabilizing agent, optionally said active compound and an aqueous phase; and
b) subjecting the components of step a) to conditions so that an isotropic, transparent and substantially homogenous preparation is formed,
wherein step b) comprises a single phase evaporation or high pressure homogenisation method.
